(19) United States
(12) Patent Application Publication
(10) Pub. No.: US 2012/0150169 A1
(43) Pub. Date: Jun. 14, 2012

(54) IMPEDANCE MEASUREMENT TO MONITOR ORGAN PERFUSION OR HEMODYNAMIC STATUS

(75) Inventors: Todd M. Zielinksi, Ham Lake, MN (US); Douglas A. Hettrick, Andover, MN (US); Avram Scheiner, Yadnais Heights, MN (US); Yong K. Cho, Maple Grove, MN (US); Mustafa Karamanoglu, Fridley, MN (US)

(73) Assignee: Medtronic, Inc.

(21) Appl. No.: 13/314,626

(22) Filed: Dec. 8, 2011

Related U.S. Application Data
(60) Provisional application No. 61/421,413, filed on Dec. 9, 2010.

(51) Int. Cl. A61B 18/00

(52) U.S. Cl. 606/34

(57) ABSTRACT

A system and method for deliverying an ablation therapy that includes delivering the ablation therapy, delivering drive signals to establish a drive signal vector fields, determining impedance signals in response to the drive signals, determining a first impedance parameter in response to the first impedance signal and a second impedance parameter in response to the second impedance signal, determining whether there is a change in a hemodynamic status of the tissue subsequent to delivery of the ablation therapy in response to the first impedance parameter and the second impedance parameter, and adjusting delivery of the ablation therapy in response to determining whether there is a change in a hemodynamic status of the tissue.

### RELATED APPLICATION

The present application claims priority and other benefits from U.S. Provisional Patent Application Ser. No. 61/421,413, filed Dec. 9, 2010, entitled “IMPEDANCE MEASUREMENT TO MONITOR ORGAN PERFUSION OR HEMODYNAMIC STATUS”, incorporated herein by reference in its entirety

### TECHNICAL FIELD

The disclosure relates generally to implantable medical devices and, in particular, to a method and apparatus for monitoring a hemodynamic status of a portion of a patient's body using impedance measurements.

### BACKGROUND

In a number of medical and surgical procedures, it is desirable to monitor the hemodynamic status of a particular organ or a local region of a patient's body. For example, it may be desirable to monitor parameters relating to the perfusion of a specific organ or body region in the course of diagnostic testing or to control the operation of an implantable device. The direct measurement of blood flow using sensors introduced into the arterial blood stream is generally not practiced in chronically implantable medical device systems because of associated risks, such as thrombus formation and bleeding. Impedance measurements have been proposed to measure signals correlated to changes in blood volume in a vessel or heart chamber. The cyclical changes in the impedance measurements are related to pulsatile blood flow through the vessel or chamber. A need remains, however, for a method and apparatus for performing clinically meaningful measurements that allow the hemodynamic status of a specific portion of a patient's body to be monitored for diagnostic or therapy control purposes.

### BRIEF DESCRIPTION OF THE DRAWINGS

FIG. 1 is a functional block diagram of an implantable medical device (IMD) for monitoring the hemodynamic status of an organ, such as the kidney, or other portion of a patient's body.

FIG. 2A is schematic view of one lead and electrode configuration for positioning impedance measuring electrodes along an artery and/or vein.

FIGS. 2B and 2C depict two different measurement techniques that could be used with the lead configuration shown in FIG. 2A.

FIG. 2D is a schematic diagram of a measurement configuration using the leads shown in FIG. 2A.

FIG. 3 is a schematic diagram of an alternative lead and electrode configuration for measuring impedance for use in monitoring blood flow into and out of an organ.

FIG. 4 is schematic diagram of an alternative lead and electrode configuration 100 for monitoring a hemodynamic condition of an organ or region of a patient's body.

FIG. 5 is a schematic diagram of another configuration of a lead and electrode arrangement for measuring impedance to monitor a hemodynamic status of an organ.

FIG. 6 is a flow chart of an impedance monitoring method according to one embodiment.

FIG. 7 is a flow chart of one method for analyzing and responding to impedance data acquired using at least two different drive current frequencies.

FIG. 8 is a flow chart of an alternative method for analyzing impedance data.

FIG. 9 is a flow chart of one method for performing impedance monitoring for facilitating delivery of ablation therapy.

FIG. 10 is a time-based plot of an arterial blood pressure signal and corresponding magnitude signal and phase angle signal of the arterial reactive admittance (the reciprocal or impedance) over four cardiac cycles.

FIG. 11 is a time-based plot of the magnitude of an arterial reactive admittance waveform and a venous reactive admittance waveform.

### DETAILED DESCRIPTION

In the following description, references are made to illustrative embodiments. It is understood that other embodiments may be utilized without departing from the scope of the disclosure. In some instances, for purposes of clarity, identical reference numbers may be used in the drawings to identify similar elements.

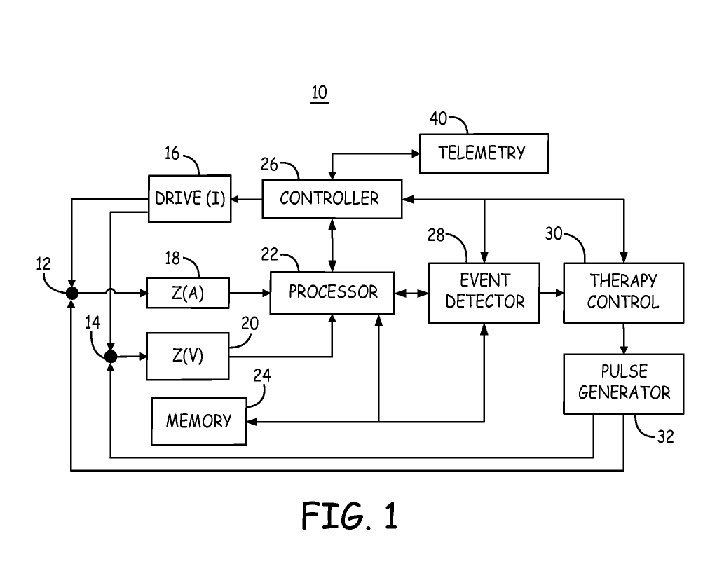

FIG. 1 is a functional block diagram of an implantable medical device (IMD) for monitoring a hemodynamic status of a portion of a patient's body, such as an organ, e.g. the kidney. IMD 10 is coupled to at least one pair of electrodes 12, 14 for measuring the impedance of at least a portion of an artery perfusing a portion of a patient's body and a vein carrying blood flowing away from the organ or portion of the patient's body.

The electrodes 12, 14 receive a drive current signal from drive signal circuit 16 under the control of controller 26. The resulting voltage signal is received by impedance measuring circuitry 18 and 20. The voltage signal may be used directly as a signal correlated to impedance changes in the artery and/or vein or for computing an impedance signal from the measured voltage (and known drive signal current). The voltage signal, used directly, and the actual impedance signal (or reciprocal of the impedance known as “admittance”) computed from a measured voltage signal are both referred to herein collectively as an “impedance signal”. The impedance signal relating is used for monitoring a hemodynamic status of an organ or portion of the patient's body and/or therapy control purposes.

In some embodiments, a single impedance signal may be obtained that relates to both arterial and venous impedance changes. In other embodiments, an impedance signal corresponding substantially to arterial impedance 18 and an impedance signal corresponding substantially to venous impedance 20 are acquired and provided to signal processor 22. The controller 26, signal processor 22, event detector 28 and other modules described herein may be embodied as an application specific integrated circuit (ASIC), an electronic circuit, a processor (shared, dedicated, or group) and memory that execute one or more software or firmware programs, a combinational logic circuit, or other suitable components that provide the described functionality.

Processor 22 uses the impedance signals received from impedance measuring circuitry 18 and 20 to compute characteristics of the impedance signals that correlate to the hemodynamic status of the organ or body portion. The measured values are provided to event detection module 28 for detecting physiological events relating to a patient condition, such as heart failure, renal failure or other organ failure or for detecting a need for adjusting a therapy delivered by the IMD.

Events detected by detector 28 and/or impedance signal information from processor 22 may be stored by memory 24. Event detector 28 in cooperation with processor 22 provides signals to controller 26 (or directly to therapy control module 30) for controlling therapy control module 30. In one embodiment, therapy control module 30 controls an electrical pulse generator 32 to deliver therapeutic electrical pulses to the patient. The pulses may be delivered using the impedance measuring electrodes 12, 14 or other electrodes coupled to IMD 10.

The pulses may be stimulating pulses delivered to cause activation of excitable tissue, such as, but not limited to, the myocardium, a baroreceptor, a vagal nerve, or a renal nerve. The pulses may alternatively be ablation energy delivered to ablate tissue in the wall of an artery, such as nervous tissue in the renal artery wall or other nervous tissue.

Therapy control 30 may additionally or alternatively control a fluid pump or other fluid delivery device for delivering a therapeutic drug or biological fluid to the patient in response to events detected by detector 28. In alternative embodiments, IMD 10 may be provided as a monitoring-only device without therapy delivery capabilities. In that case, IMD 10 may be in telemetric wired or wireless communication with another device delivering a therapy to the patient. The therapy delivery device may control a therapy delivered to the patient using the impedance-related data received from IMD 10.

Controller 26 is further shown coupled to a telemetry module 30 which includes telemetry circuitry and an antenna for transmitting data acquired by IMD 10 to an external programmer or home monitoring device and to receive programming data or commands from an external device. Telemetry module 30 may be used for issuing a warning or alert to the patient or a clinician in response to detecting an event or condition relating to the hemodynamic status of the monitored organ or body portion.

While not explicitly shown in FIG. 1, it is recognized that IMD 10 may operate in conjunction with other sensors, including for example ECG or intracardiac electrogram (EGM) sensing electrodes for sensing cardiac signals such as R-waves or P-waves, a pressure sensor, accelerometer, flow sensor, blood oxygen sensor, temperature sensor or the like. Other physiological signals may be used in combination with the impedance signals for detecting physiological events and for controlling a therapy.

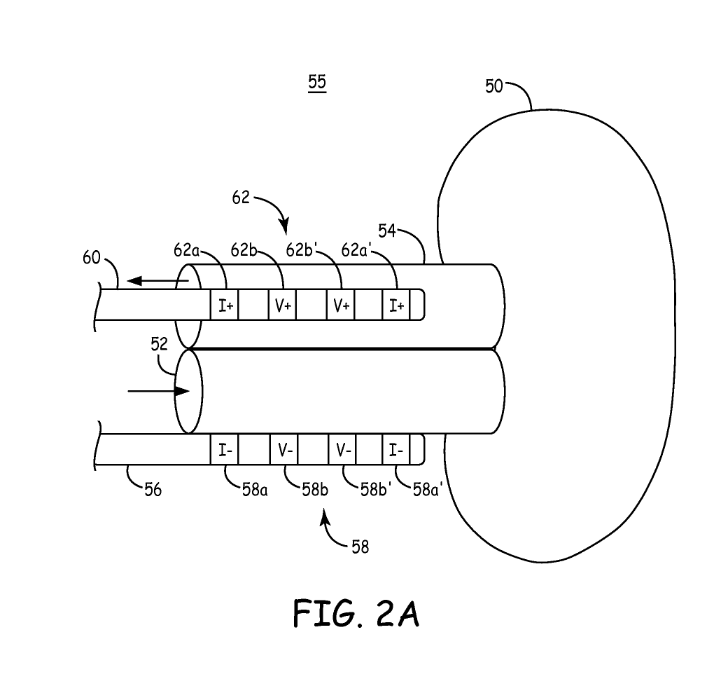

FIG. 2A is schematic view of one lead and electrode configuration 55 for positioning impedance measuring electrodes along an artery and/or vein. A renal artery 52 and a renal vein 54 are shown schematically, providing blood flow respectively into and out of a patient's kidney 50. A first electrode array 58 is carried by an electrical lead 56 positioned in a paravascular location adjacent to or in direct contact with the renal artery 52. The electrode array 58 includes a pair of drive current electrodes 58a, 58a′ and a pair of measurement or recording electrodes 58b, 58b′. The paravascular positioning of the lead 56 allows an impedance signal responsive to changes in the blood volume in artery 52 during a cardiac cycle to be recorded without requiring a lead located in the arterial bloodstream.

A second electrode array 62 is carried by a second lead 60 which is advanced intravenously into the renal vein 54. The second array 62 includes a pair of drive current electrodes 62a, 62a′ and a pair of recording electrodes 62b, 62b′. The second lead 60 may alternatively be positioned in a paravascular location directly adjacent to the vein 54. In a paravascular position, either of leads 56 and 60 may be positioned in direct contact with the corresponding vessel by advancing the portion of the lead carrying the electrode array within the adventitial layer surrounding artery 52 or vein 54, respectively. Alternatively, the electrodes 58 and 62 may be adjacent to the respective artery or vein with adventitial tissue and possibly other tissue therebetween.

The drive current electrodes 58a and 58a′ are shown having a negative polarity and the drive current electrodes 62a and 62a′ are shown having a positive polarity. A drive current signal may be applied across the drive current electrode pairs to produce a current field traversing renal artery 52 and a portion of vein 54. In this example, recording electrodes 58b, 58b′ are shown having a negative polarity and recording electrodes 62b, 62b′ are shown having a positive polarity. A voltage signal measured between a para-arterial electrode 58b and an intravenous electrode 62b will provide a signal that may have both arterial and venous contributions. As will be described below, arterial and venous impedance information may be obtained for separate analysis by controlling the frequency of a drive current signal and/or in the selection of the electrodes used for delivering the drive current and for recording an impedance signal.

While each lead 56 and 60 are shown carrying four electrodes, the leads may be provided with more than or less than four electrodes in other embodiments. As the number of electrodes is increased, more electrodes are available for selection in particular combinations for obtaining different current vector fields and recording vectors. Generally, the more spaced out the recording electrodes, the larger the measurement volume. A more localized impedance measurement can be obtained by selecting recording electrodes closer together.

According to one use of the electrode configuration 55, a tip electrode 62a′ and a second tip electrode 58a′ may be selected as the drive current electrode pair. Electrodes 62b′ and 58b′ may be selected as the measurement electrode pair. A lowest impedance path through the target organ 50 will allow an impedance measurement to include arterial, venous and capillary bed volumes encompassing a portion of organ 50.

FIGS. 2B and 2C depict two different measurement techniques that could be used with the lead configuration shown in FIG. 2A. In FIG. 2B, a transverse current field 70 is established between electrodes 62a and 58a which are substantially in radial opposition to each other. A voltage signal is measured between opposing electrodes 62b and 58b such that the measurement vector field 72 traverses the artery 52 and at least a portion of vein 54 in a substantially transverse radial manner.

As shown in FIG. 2C, in order to obtain a measurement vector field that is more longitudinally directed than the substantially radial field 72, electrodes 62a and 58a′, which are displaced from each other longitudinally may be used to establish a drive current field 74. A measurement vector field 76 between electrodes 62b and 58b′, which are longitudinally displaced from each other, provides a relatively more longitudinal measurement vector field 76.

FIG. 2D is a schematic diagram of a measurement configuration using the leads 56 and 60 shown in FIG. 2A. The drive current field may be established using two electrodes, 58a, 58a′ or 62a, 62a′, along the same lead 56 or 60, respectively. The induced voltage signal may then be measured using two electrodes 58b, 58b′ or 62b, 62b′ along the same respective lead 56 or 60. In this way, a drive current field and measurement vector field are substantially aligned longitudinally with the blood vessel the lead is associated with. When arterial impedance measurements are made, only lead 56 is used and when venous measurements are made, only lead 60 is used.

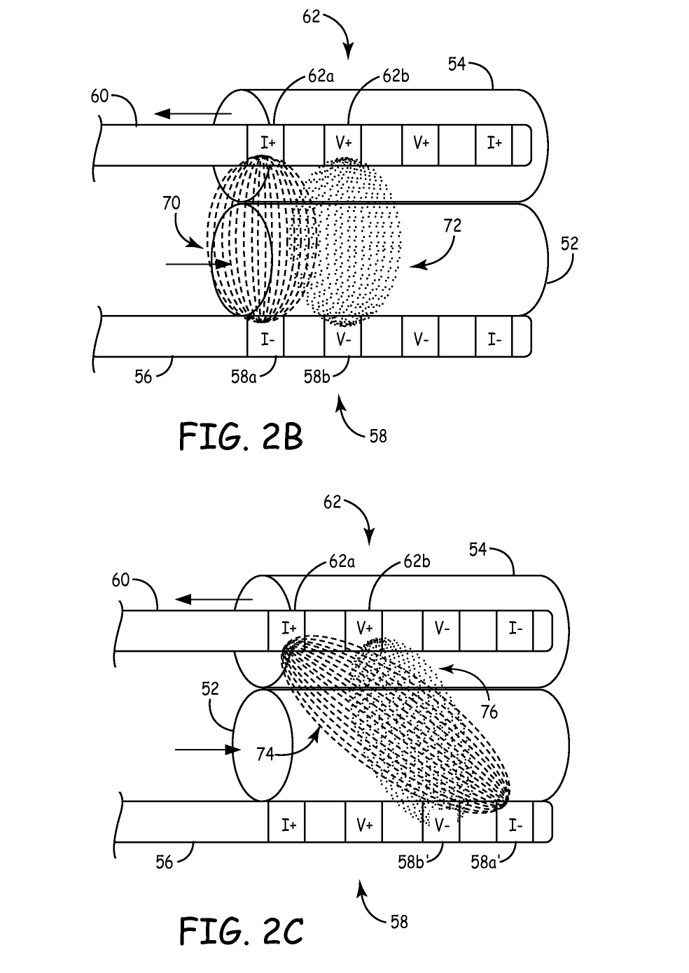

FIG. 3 is a schematic diagram of an alternative lead and electrode configuration 80 for measuring impedance for use in monitoring blood flow into and out of an organ. An artery 84 and vein 82 entering and exiting an organ commonly extend in close proximity with each other. A lead 81 carries electrodes 86 from which a drive current electrode pair and a measurement electrode pair can be selected. Lead 81 may be positioned between the vein 82 and artery 84 such that the impedance signal includes contributions from both an arterial blood volume and a venous blood volume as shown by the measurement vector field 88, illustrated in FIG. 3.

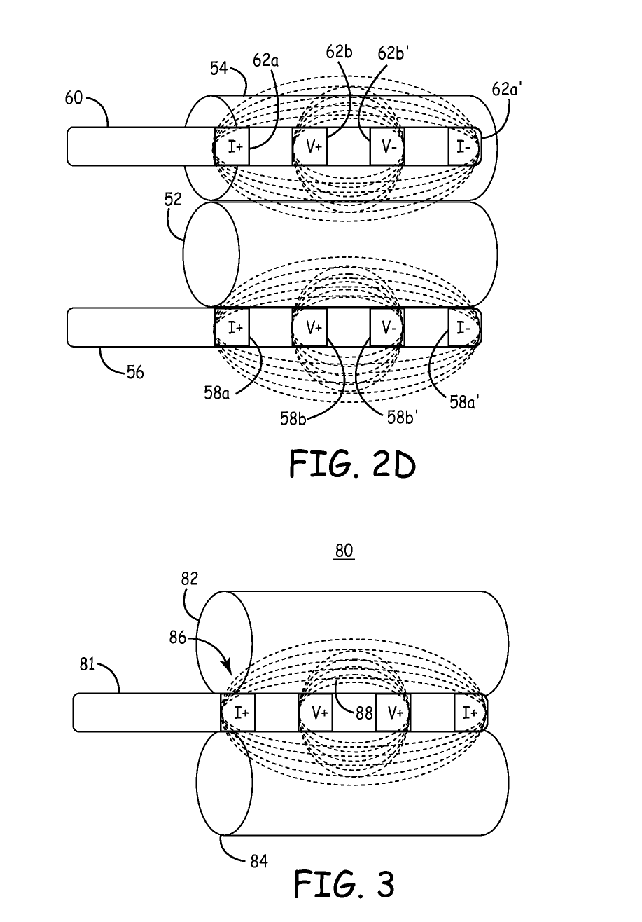

FIG. 4 is a schematic diagram of an alternative lead and electrode configuration 100 for monitoring a condition of an organ or region of a patient's body. In this example, an organ 110, which may be a kidney, is shown with an artery 106 and vein 108 providing blood flow into and out of organ 110. A transvenous lead 102 is positioned within the vein 108 and carries at least two electrodes 104 that can be selected for use in delivering a drive current and/or measuring an impedance signal. A subcutaneous lead 112 carrying at least one electrode 114 is positioned relative to the transvenous lead 102 such that organ 110 is located therebetween. The subcutaneous electrode may be positioned under the skin, under muscular tissue or within a cavity, e.g. the abdominal or thoracic cavity, of the patient.

A drive current vector field 116 or 116′ and a measurement vector field 118 or 118′ can be established using selected drive current and measurement electrode pairs each having at least one electrode on lead 102 and one electrode on lead 112. A measurement vector field 118 or 118′ extending between intravenous lead 102 and extravascular lead 112 will encompass a portion of the target organ, including arterial, venous, and capillary volumes.

In other embodiments, two extravasular leads may be positioned with a target organ or body tissue therebetween to obtain a measurement vector field extending across the target organ or tissue region. The leads may be positioned in close adjacent proximity to the organ or at a greater distance from the target organ or tissue, depending on how localized the impedance measurements need to be and anatomical and surgical access.

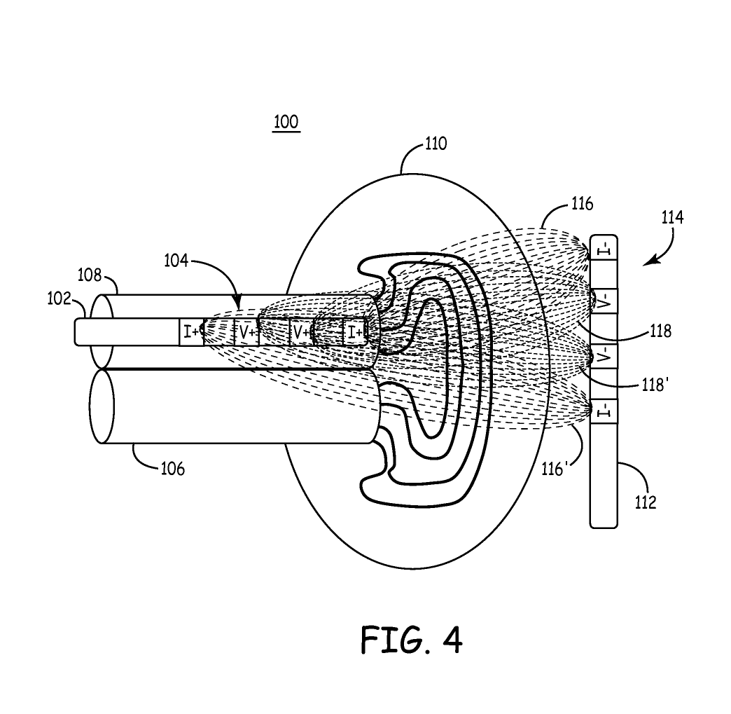

FIG. 5 is a schematic diagram of another configuration 150 of a lead and electrode arrangement for measuring impedance to monitor an organ condition. The target organ 158, shown here as a kidney, is perfused by an artery 154 and exiting vein 152 and innervated by renal nerve 156. A lead 160 may be configured with a flexible cuff 162 carrying multiple electrodes (not shown). Cuff 162 is sized to wrap around the vein 152, artery 154 and nerve 156. In this way, electrodes positioned along an inner surface of cuff 162 can be selected to deliver a drive signal or measure an impedance signal that will include both arterial and venous blood volume contributions.

Additionally, the electrodes positioned along cuff 162 may be used to deliver therapeutic electrical stimulation. In this example, the stimulation may be an ablative energy for ablating renal nerve tissue. In other implant locations associated with other targeted blood vessels an electrical stimulation therapy may be delivered, for example, to a carotid baroreceptor or vagal nerve. It is to be understood that in the various lead and electrode configurations shown and described herein, electrodes available for performing impedance measurements may also be used for therapy delivery.

It is further recognized that a cuff electrode may be configured to surround a vein only, an artery only, a nerve and vein, a nerve and artery, or all three as shown in FIG. 5. More than one cuff electrode may be implanted. One cuff may be surrounding a vein and one surrounding an artery to allow separate venous and arterial impedance measurements.

It is recognized that with multiple electrodes positioned along the leads as presented herein, multiple configurations of the drive current vector field and measurement vector field can be obtained through the selection of different pairs of electrodes and polarity assignments. Drive current electrode pairs and recording electrode pairs may be selected along the same or different leads. Electrode spacing and position along a given lead may vary between embodiments. In some embodiments, a drive current pair and a recording pair may share a common electrode so that less than four electrodes can be used to measure an impedance signal.

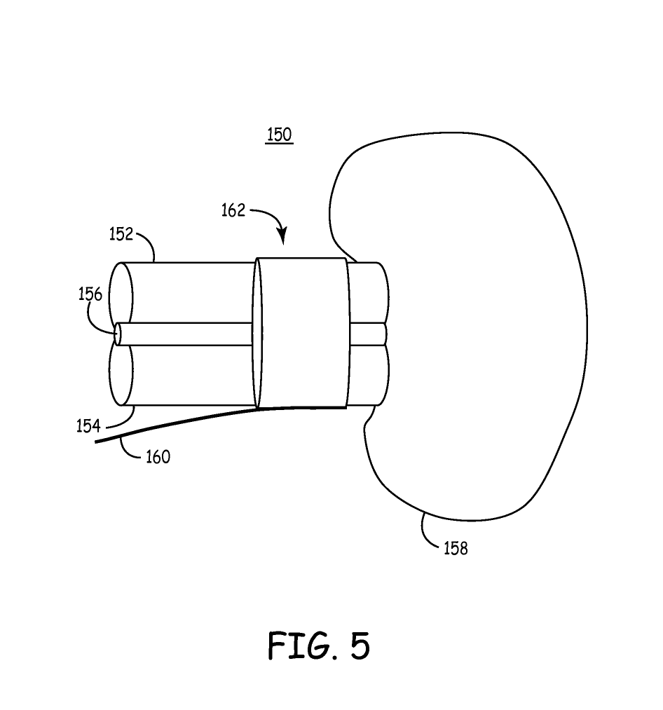

FIG. 6 is a flow chart 200 of an impedance monitoring method according to one embodiment. Flow chart 200 and other flow charts presented herein are intended to illustrate the functional operation of the device, and should not be construed as reflective of a specific form of software or hardware necessary to practice the methods described. It is believed that the particular form of software will be determined primarily by the particular system architecture employed in the device and by the particular detection and therapy delivery methodologies employed by the device. Providing software to accomplish the described functionality in the context of any modern IMD, given the disclosure herein, is within the abilities of one of skill in the art.

Methods described in conjunction with flow charts presented herein may be implemented in a computer-readable medium that includes instructions for causing a programmable processor to carry out the methods described. A “computer-readable medium” includes but is not limited to any volatile or non-volatile media, such as a RAM, ROM, CD-ROM, NVRAM, EEPROM, flash memory, and the like. The instructions may be implemented as one or more software modules, which may be executed by themselves or in combination with other software.

At block 202, the impedance monitoring is initiated, which may occur at the time the IMD is implanted or at a later time in response to a programming instruction entered by a user. At block 204, a monitoring schedule is established. The monitoring schedule may include a recording window, a monitoring time period and a monitoring frequency.

The recording window defines the duration of time that an impedance signal is recorded. For example a number of seconds, minutes, or number of cardiac cycles may be defined as the recording duration window. The monitoring frequency is the number of times the recording window is applied over the monitoring time period. For example, if the monitoring time period is one day, the monitoring frequency may be hourly meaning that each hour over the course of one day the impedance signal is recorded for the duration of the recording window. This monitoring schedule would allow daily averages of impedance signal characteristics to be determined and trends in daily measurements to be determined over time.

In another illustrative example, the monitoring period may be one week with a monitoring frequency of once per day and a measurement window of one minute. In this case, impedance signals will be recorded daily for one minute and weekly averages may be determined. It is recognized that numerous monitoring schedules may be established at block 202 and will depend on the particular monitoring application and patient needs. The recording window used for signal acquisition and the monitoring frequency may be range from seconds to days, weeks or more. These monitoring control parameters may be set to initial default values or may be established at block 202 by clinician programming. The monitoring schedule may be adjusted automatically by the IMD based on changes in measured impedance signals, changes in other physiological signals being monitored by the IMD, or changes in a delivered therapy.

At block 204 measured parameter threshold levels are established. The threshold levels may be established for generating a patient or clinician warning and/or for causing an adjustment to a therapy, which may include turning a therapy on or off or adjusting a therapy delivery control parameter. Parameter threshold levels may be set at default values and may be programmable by a clinician.

At block 205, the desired electrode vector configuration and electrode polarity assignments are selected for delivering the drive current and recording the impedance signal. In some embodiments, multiple leads and multiple electrodes may be available to select one or more impedance recording configurations by selecting different drive signal and recording electrode combinations.

At block 206, the drive current amplitude is selected. Selection of the drive current amplitude will depend on the selected vector configuration and the particular monitoring application. Initially, a drive current frequency may be set to low at block 208. Application of different drive current frequencies is used to obtain different impedance signals. Differences in arterial and venous anatomy may result in different impedance signal responses to different drive current frequencies. The arterial wall is characterized by a thicker smooth muscle layer than veins. Arteries present in the impedance measurement vector field are expected to produce a higher reactive impedance component. Veins are expected to produce a higher real impedance component that is less frequency dependent than the reactive impedance component produced by the arteries.

An initial low frequency drive signal applied at block 208 may be approximately 10 kHz or less in one embodiment. In accordance with the established monitoring schedule, the impedance signal response to the low frequency drive signal is recorded at block 210. Characteristics of the resulting impedance signal may be analyzed to evaluate a hemodynamic status relating to venous compliance, venous blood pressure, or venous blood flow. Various signal characteristics that may be extracted and used for monitoring venous properties or venous-related hemodynamics at the targeted monitoring site will be further described below. The impedance signal may be sampled and stored at block 210 as a digitized signal for later analysis.

Alternatively, the signal may be buffered over each cardiac cycle or over the recording window with selected points of the impedance signal extracted in real time and stored in device memory for later analysis. In one embodiment, the maximum and minimum magnitude and phase angle of the impedance signal during each cardiac cycle during the recording window are stored. An average impedance magnitude and phase angle over the recording window may be determined and stored. An area corresponding to the impedance magnitude and phase angle waveform or an integral of the magnitude or phase angle waveform may be determined for each cardiac cycle or a portion thereof.

At block 212, the drive signal is set to a high frequency, for example approximately 100 kHz or higher. The resulting impedance signal is recorded at block 214 in accordance with the impedance monitoring schedule and will be used to extract signal characteristics useful in monitoring arterial compliance, pressure or blood flow. The impedance signal may be sampled and stored for later analysis or buffered during the recording window to allow selected features to be extracted and stored, such as the cardiac cycle maximum, minimum and area of the impedance magnitude and phase the average magnitude and phase angle over the recording window as described above. Analysis of impedance signal characteristics will be further described below.

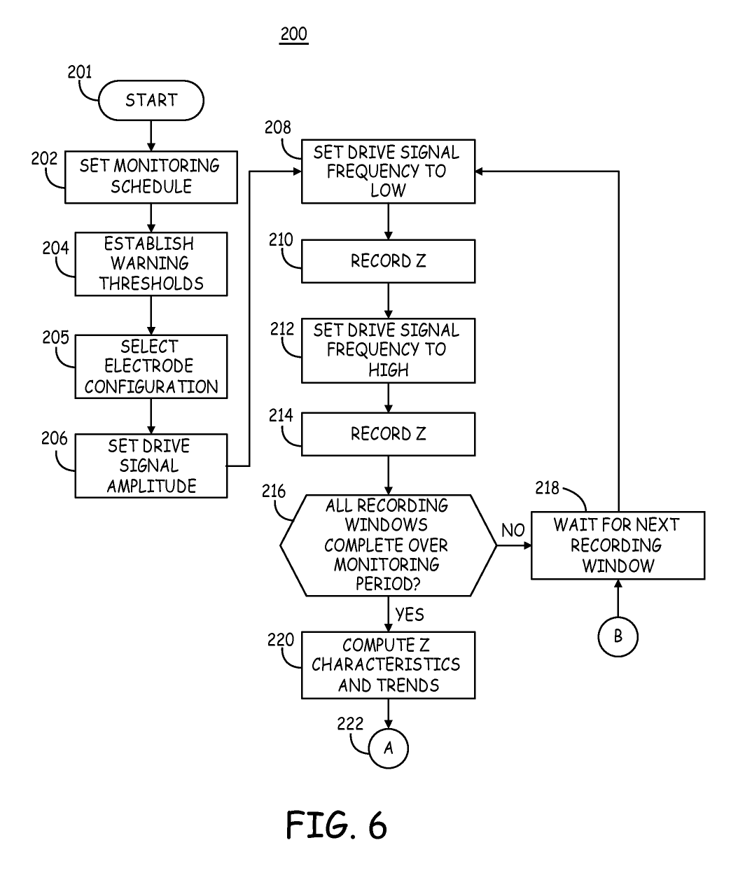

If all recording windows have been completed as scheduled over a desired monitoring period, the impedance characteristics and trends for the monitoring period are computed at block 220. If the monitoring period has not expired, the process advances to block 218 to wait for the next scheduled recording window. Once the monitoring period is complete, the process advances to flow chart 300 (FIG. 7) as indicated by connector A 222.

While the different frequency drive signals are described in the foregoing as being delivered sequentially, in other embodiments two different frequency drive signals may be delivered simultaneously, to the same or different electrodes. The frequencies should have a minimum range separation of, for example, at least 50 kHz. Examples of simultaneous drive signal frequencies may be 5 kHz and 50 kHz or 10 kHz and 100 kHz though numerous combinations are possible. The drive signals having two different frequencies may also be delivered to the same set of electrodes then measured using two different recording electrode pairs. Alternatively, two different drive signal frequencies may be delivered using the same electrode pair that is used to record the impedance signals using a filter to separate the two signal responses.

FIG. 7 is a flow chart 300 of one method for analyzing and responding to impedance values acquired using at least two different drive current frequencies. At block 302, selected time intervals between fiducial points extracted from the impedance signal may be measured in addition to the magnitude and phase angle measurements described previously. For example, intervals measured at block 302 may include the time difference between the maximum magnitude and maximum phase angle, the minimum magnitude and minimum phase angle, a maximum or minimum magnitude and a preceding or subsequent R-wave, and a maximum or minimum phase angle and a preceding or subsequent R-wave, or any combination thereof.

At block 304, averages and/or trends of any of the magnitude, phase angle, and time interval measurements obtained over the monitoring period are computed. For example, a daily trend in the any of the measured parameters may be computed at block 304 by comparing an average measurement obtained from a recording window to a previous recording window. Chronic trends may be computed at block 306 by comparing monitoring period measurements or averages to previous monitoring period measurements or averages or by comparing a measurement obtained from a given recording window to a measurement obtained from a previous recording window applied at the same time of day during an earlier monitoring period.

At block 308, measurements and/or trends obtained from the most recent monitoring period and/or chronic measurement trends (obtained across more than one monitoring period) are compared to respective established thresholds. If a threshold crossing is detected at block 308, a warning may be generated at block 310. A warning may be transmitted to an external device for display on a programmer, home monitoring device, remote patient monitoring system, or the like. Alternatively, a warning may be an audible sound or perceptible vibration or stimulation delivered to the patient. Additionally or alternatively, if a need for therapy is detected at decision block 311, based upon the threshold crossing, a therapy delivered by the IMD may be turned on or otherwise adjusted at block 312 (or in some cases turned off).

For example, a cardiac pacing therapy or a neurostimulation therapy using electrodes located at a different anatomical site than the impedance monitoring electrodes may be adjusted in response to detecting a threshold crossing in an attempt to improve the blood flow to the monitored organ or body region. In some embodiments, the electrodes available for monitoring impedance are also used in delivering a therapy that is adjusted at block 312. Such therapies may include a nerve stimulation therapy or autonomic receptor stimulation such as baroreceptor stimulation. After adjusting a therapy, the monitoring method may return to block 218 as indicated by connector B 314 to wait for the next recording window. The impedance response during the next recording window or next monitoring period may be used to make further adjustments to a therapy as needed.

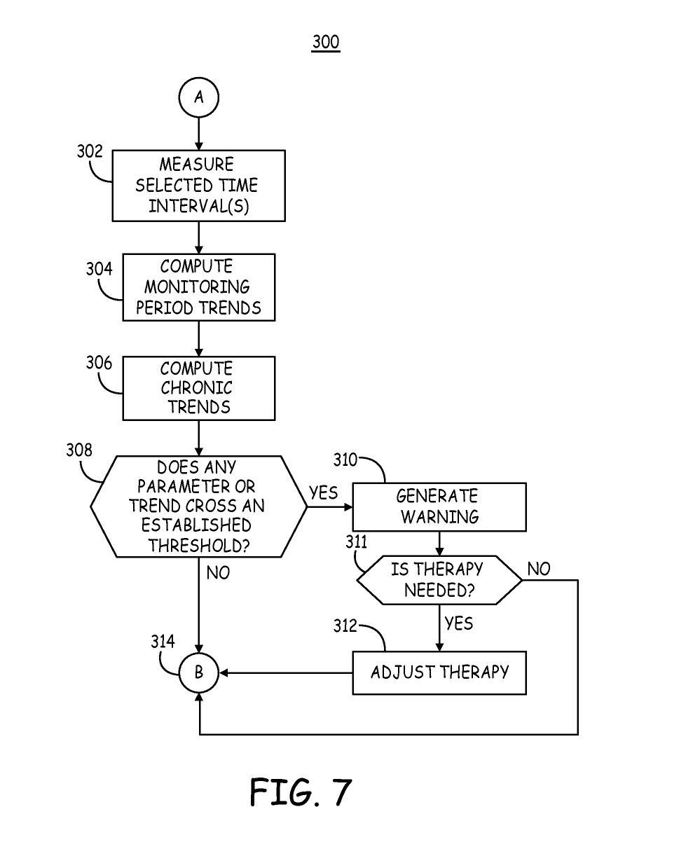

FIG. 8 is a flow chart 400 of an alternative method for analyzing impedance data. The process shown in flow chart 200 (FIG. 6) may advance from connector A 222 to A′ 401 to measure selected time intervals at block 402 as described above. At block 404, differences between parameters measured from the high-frequency response arterial signal and the low-frequency response venous signal may be computed. The impedance signal recorded in response to the high frequency drive signal is considered to be more highly correlated to arterial impedance. The impedance signal recorded in response to the low frequency drive signal is considered to be highly correlated to venous impedance. Differences in analogous parameters determined from the two signals, e.g., maximum magnitude, maximum phase angle, or the like, may be determined as arterial-venous differences at block 404.

A trend in an arterial-venous difference is computed at block 406. A trend may be determined over a given monitoring period or over multiple monitoring periods. A change in the difference between an arterial related impedance parameter and a venous related impedance parameter, i.e. a difference in the trends in an arterial related parameter and a venous related, parameter may be indicative of a change in perfusion of the target organ or body region perfused by vessels being monitored. If, for example, a given parameter is increasing proportionally for both the arterial-related signal and the venous-related signal and then begins to decrease for one of the arterial or venous signals while continuing to increase for the other signal, a change in the trend of the arterial-venous difference for that parameter is detected. A change in the relative difference between an arterial and venous impedance parameter may indicate organ failure or other adverse condition.

At block 408, an arterial-venous difference in any of the measured impedance signal parameters or changes in the relative trends in the arterial- and venous-related parameters may be compared to a threshold. If a threshold crossing is detected, a clinician or patient warning may be generated at block 410 and/or a therapy may be adjusted automatically by the IMD at block 412 after determining a need for a therapy adjustment at block 411 based on the threshold crossing.

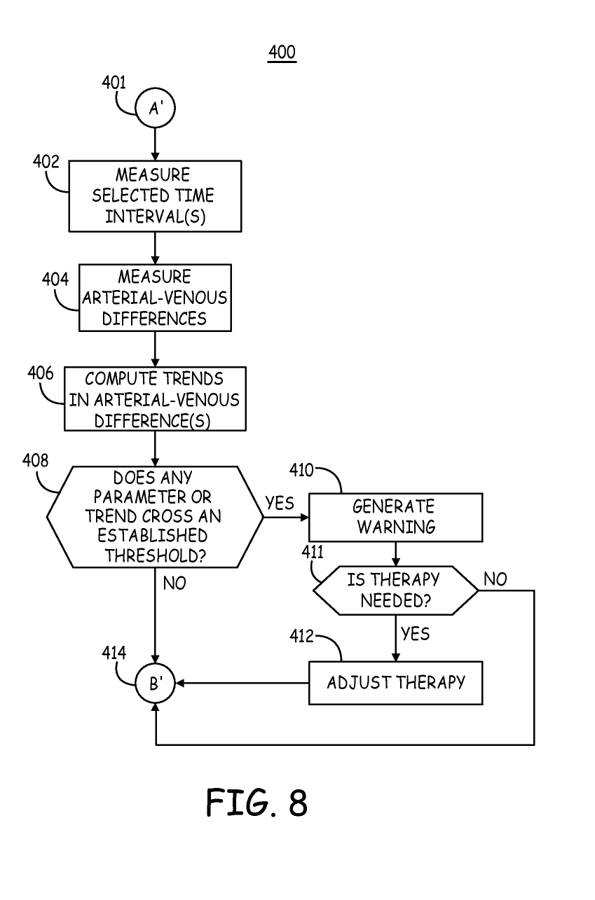

FIG. 9 is a flow chart 500 of one method for performing impedance monitoring for facilitating delivery of ablation therapy. At block 501, an impedance monitoring catheter is positioned in an intravenous or paravascular location for monitoring impedance. Any of the configurations described above may be used for monitoring impedance for facilitating ablation therapy. In one embodiment, an impedance monitoring/ablatation catheter is advanced intra-arterially or para-arterially to position electrodes adjacent the renal artery. At least one pair of electrodes carried by the catheter is coupled to a drive current source and one pair is coupled to an impedance monitoring circuitry as shown in FIG. 1. The drive current electrode pair and the recording electrode pair may share common electrodes in some embodiments.

At least one electrode pair carried by the lead, which may the same or a different pair than the drive current or recording electrode pairs, is coupled to an ablative energy source, which may correspond to the pulse generator 32 shown in FIG. 1. Referring to FIG. 2A, for example, electrodes 58a and 58a′ may be used to deliver a drive current signal. Electrodes 58a and 58b′ may be used to measure an impedance signal. Electrode 58b may be coupled to an ablative energy source using the common electrode 58a to deliver an ablation therapy. Ablation energy may be delivered for renal sympathetic nerve ablation for treatment of hypertension. In other applications, the lead(s) may be positioned to allow ablation of sympathetic nerves in other body locations.

The electrodes are used to obtain baseline impedance measurements at block 502. Any of the impedance measurements described above may be used in computing an impedance metric correlated to blood pressure for obtaining a baseline measurement. A high frequency drive current signal may be delivered to obtain an arterial-related impedance signal alone or in combination with a low frequency drive current signal delivered to determine a venous component as described above. Since the arterial information may be of particular interest, determination of the venous contribution to an arterial signal may be determined using a low frequency drive signal and adjusting (or subtracting) the venous contribution from the high frequency, arterial-related impedance response.

After obtaining the baseline signal, ablation therapy is delivered using the same or different electrodes along the monitoring/therapy catheter at block 504. At block 506, post-therapy impedance measurements are obtained. The post-therapy measurements are compared to the baseline measurements at block 508 to determine if an expected or desired change in the impedance measurement has occurred. If not, ablation energy may be delivered again at block 504, using the same or different electrodes than the first ablation delivery. When a desired change in the impedance-based metric of arterial blood pressure has been achieved, as determined at block 508, the procedure is stopped at block 510.

The monitoring catheter may remain in place for continued follow-up and chronic monitoring of impedance signals. If the ablation procedure does not produce a chronically effective decrease in systemic blood pressure, the catheter may be used again to repeat an ablation procedure to obtain a better result.

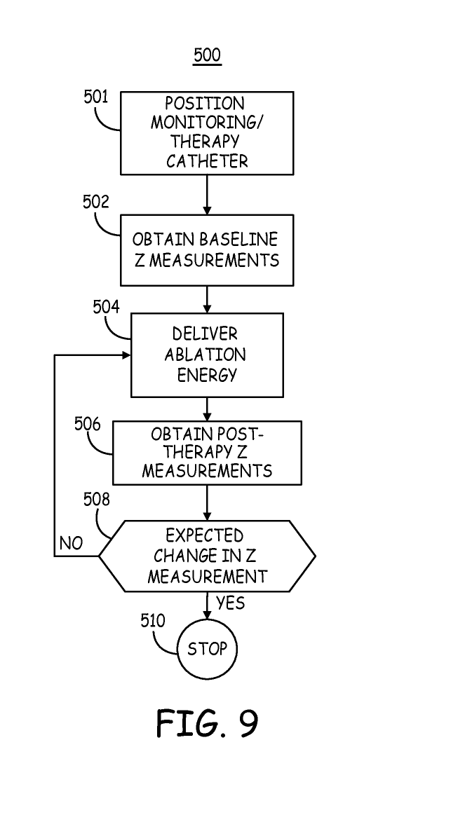

FIG. 10 is a time-based plot 550 of an arterial blood pressure signal 552 and corresponding magnitude signal 554 and phase angle signal 556 of the arterial reactive admittance (the reciprocal of impedance) over several cardiac cycles 558. As can be seen, the pulsatility of both the admittance magnitude signal and the admittance phase angle is well-correlated (directly and inversely, respectively) to the arterial blood pressure signal 552.

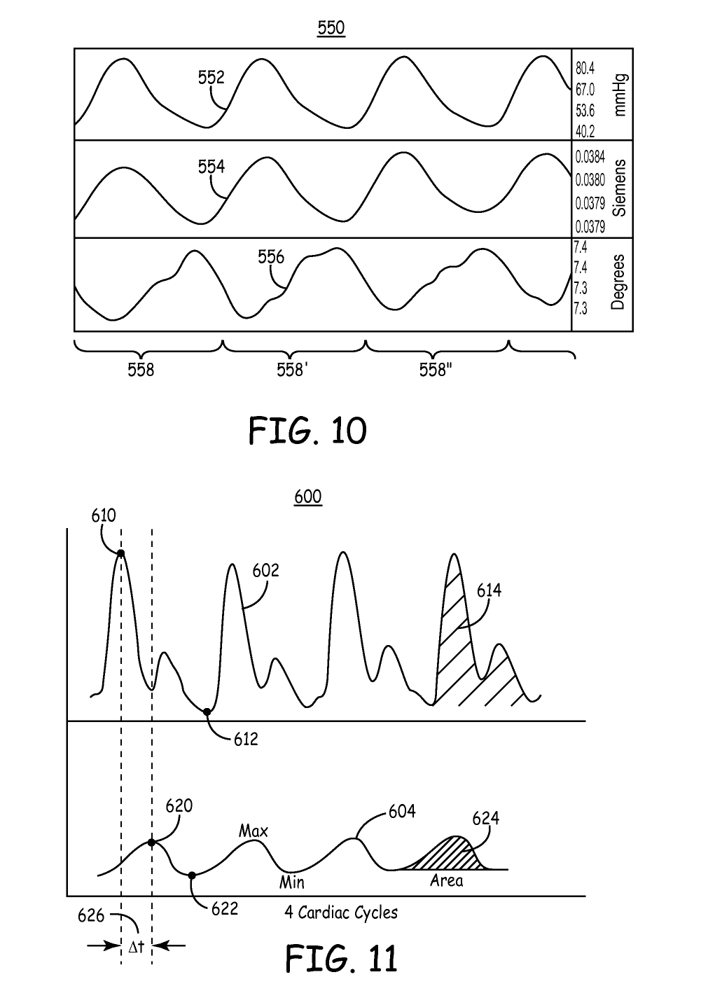

FIG. 11 is a time-based plot 600 of the magnitude of an arterial reactive admittance waveform 602 and a venous reactive admittance waveform 604. The arterial waveform is measured in response to a drive signal frequency of 100 kHz or more. The venous waveform is measured in response to a drive signal frequency of 10 kHz or less. Various signal admittance signal characteristics that may be measured include the maximum magnitudes 610, 620 of the arterial and venous waveforms, respectively, the minimum magnitudes 612, 622, an area or integral of the waveform over one cardiac cycle 614, 624, and time intervals such as the time difference 626 between the maximum magnitude 610 of the arterial waveform and the maximum magnitude 620 of the venous waveform.

It is recognized that numerous attributes of the impedance (or admittance) waveform may be measured for use in deriving a metric correlated to blood pressure, blood flow or vessel compliance and more generally the hemodynamic status of the local portion of the patient's body. The signal features shown in FIG. 11 are illustrative and not intended to be limiting as to the types of signal features or characteristics that may be used in determining a value that is used as a metric of a hemodynamic status of the targeted organ or portion of the patient's body. Other features may include a slope measurement, mean signal amplitude, and peak-to-peak differences.

Thus, a system and method for monitoring impedance have been presented in the foregoing description with reference to specific embodiments. It is appreciated that various modifications to the referenced embodiments may be made without departing from the scope of the disclosure as set forth in the following claims.

1. A method for deliverying an ablation therapy to a tissue of a patient's body, comprising:
delivering the ablation therapy to the tissue;
delivering a first drive signal having a first frequency to establish a first drive signal vector field;
determining a first impedance signal in response to the first drive signal;
delivering a second drive signal having a second frequency different than the first frequency to establish a second drive signal vector;
determining a second impedance signal in response to the second drive signal;
determining a first impedance parameter in response to the first impedance signal and a second impedance parameter in response to the second impedance signal;
determining whether there is a change in a hemodynamic status of the tissue subsequent to delivery of the ablation therapy in response to the first impedance parameter and the second impedance parameter; and
adjusting delivery of the ablation therapy in response to determining whether there is a change in a hemodynamic status of the tissue.

2. The method of claim 1, wherein the first drive signal vector field and the second drive signal vector field each comprise an arterial volume and a venous volume corresponding to the tissue.

3. The method of claim 1, wherein adjusting delivery of the ablation therapy comprises one of re-delivering the ablation therapy in response to a change in the hemodynamic status being determined and ceasing delivery of the ablation therapy in response to a change in the hemodynamic status not being determined.

4. The method of claim 3, wherein determining a change in a hemodynamic status comprises:
comparing the first impedance parameter and the second impedance parameter to a predetermined threshold; and
determining whether there is a change in the hemodynamic status in response to the comparing.

5. The method of claim 1, wherein determining the first impedance parameter and determining the second impedance parameter comprises determining one of a maximum magnitude, a minimum magnitude, an area corresponding to an impedance waveform, and an integral of an impedance waveform.

6. The method of claim 1, wherein determining the first impedance parameter and determining the second impedance parameter comprises determining one of a maximum phase angle, a minimum phase angle, an area corresponding to a phase angle waveform, and an integral of a phase angle waveform.

7. The method of claim 1, wherein determining the first impedance parameter and determining the second impedance parameter comprises determining a time interval between an event on the first impedance signal and an event on the second impedance signal.

8. The method of claim 1, further comprising sensing a cardiac electrical signal, wherein determining the first impedance parameter and determining the second impedance parameter comprises determining a time interval between an event on the cardiac electrical signal and an event on one of the first impedance signal and the second impedance signal.

9. The method of claim 1, wherein at least one of the first impedance signal and the second impedance signal is measured using an electrode positioned in a paravascular location.

10. The method of claim 1, wherein at least one of the first impedance signal and the second impedance signal is measured using an electrode positioned intravenously.

11. A medical device system for delivering an ablation therapy to a tissue of a patient's body, comprising:
an ablation delivery device to deliver the ablation therapy to the tissue;
a plurality of electrodes for delivering a drive signal and receiving a resulting impedance signal;
a drive signal circuit to deliver a first drive signal having a first frequency to establish a first drive signal vector field and a second drive signal having a second frequency different than the first frequency to establish a second drive signal vector;
an impedance measure module to determine a first impedance signal in response to the first drive signal and a second impedance signal in response to the second drive signal; and
a processor configured to determine a first impedance parameter in response to the first impedance signal and a second impedance parameter in response to the second impedance signal, to determine whether there is a change in a hemodynamic status of the tissue subsequent to delivery of the ablation therapy in response to the first impedance parameter and the second impedance parameter, and to adjust delivery of the ablation therapy in response to determining whether there is a change in a hemodynamic status of the tissue.

12. The system of claim 11, wherein the first drive signal vector field and the second drive signal vector field each comprise an arterial volume and a venous volume corresponding to the tissue.

13. The system of claim 11, wherein adjusting delivery of the ablation therapy comprises one of re-delivering the ablation therapy in response to a change in the hemodynamic status being determined and ceasing delivery of the ablation therapy in response to a change in the hemodynamic status not being determined.

14. The system of claim 13, wherein determining whether there is a change in a hemodynamic status comprises:
comparing the first impedance parameter and the second impedance parameter to a predetermined threshold; and
determining the change in the hemodynamic status in response to the comparing.

15. The system of claim 11, wherein determining the first impedance parameter and determining the second impedance parameter comprises determining one of a maximum magnitude, a minimum magnitude, an area corresponding to an impedance waveform, and an integral of an impedance waveform.

16. The system of claim 11, wherein determining the first impedance parameter and determining the second impedance parameter comprises determining one of a maximum phase angle, a minimum phase angle, an area corresponding to a phase angle waveform, and an integral of a phase angle waveform.

17. The system of claim 11, wherein determining the first impedance parameter and determining the second impedance parameter comprises determining a time interval between an event on the first impedance signal and an event on the second impedance signal.

18. The system of claim 11, further comprising an event detector to sense a cardiac electrical signal, wherein determining the first impedance parameter and determining the second impedance parameter comprises determining a time interval between an event on the sensed cardiac electrical signal and an event on one of the first impedance signal and the second impedance signal.

19. The system of claim 11, wherein at least one of the first impedance signal and the second impedance signal is measured using an electrode of the plurality of electrodes positioned in a paravascular location.

20. The system of claim 11, wherein at least one of the first impedance signal and the second impedance signal is measured using an electrode of the plurality of electrodes positioned intravenously.

21. A computer-readable medium storing a set of computer-executable instructions for performing a method for deliverying an ablation therapy to a tissue of a patient's body, the method comprising:
delivering the ablation therapy to the tissue;
delivering a first drive signal having a first frequency to establish a first drive signal vector field;
determining a first impedance signal in response to the first drive signal;
delivering a second drive signal having a second frequency different than the first frequency to establish a second drive signal vector;
determining a second impedance signal in response to the second drive signal;
determining a first impedance parameter in response to the first impedance signal and a second impedance parameter in response to the second impedance signal;
determining whether there is a change in a hemodynamic status of the tissue subsequent to delivery of the ablation therapy in response to the first impedance parameter and the second impedance parameter; and
adjusting delivery of the ablation therapy in response to determining whether there is a change in a hemodynamic status of the tissue.
